SEQUENCE LISTING (20 sequences)

SEQ ID NO: 1 - protein, 595 aa, synthetic construct
MALPVTALLLPLALLLHAARPEVQLVESGGGLVKPGGSRKLSCAASGFTFSDHGMHWVRQAPEKGLEWVAYISSGSSTIYYADTVKGRFTISRDNAKNTLFLQMTSLRSEDTAMYYCARSYIPYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 2 - protein, 590 aa, synthetic construct
MALPVTALLLPLALLLHAARPEVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSGGGGSGGGGSGGGGSSYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLSTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 3 - protein, 596 aa, synthetic construct
MALPVTALLLPLALLLHAARPEVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSGGGGSGGGGSGGGGSDIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEITTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 4 - protein, 782 aa, synthetic construct
MALPVTALLLPLALLLHAARPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 5 - protein, 700 aa, synthetic construct
MALPVTALLLPLALLLHAARPASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTTTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 6 - protein, 608 aa, synthetic construct
MALPVTALLLPLALLLHAARPAEVQLLESGGGLVQPGGSLRLSCAASGFTFSSYAMSWVRQAPGKGLEWVSNISSAGKGLEWVSSIDDSGASTYYADSVKGRFTISRDNSKNTLYLQMNSLRAEDTAVYYCAKDSASFDYWGQGTLVTVSSGGGGSGGGGSGGGGSTDIQMTQSPSSLSASVGDRVTITCRASQSISSYLNWYQQKPGKAPKLLIYTASSLQSGVPSRFSGSGSGTDFTLTISSLQPEDFATYYCQQSAASPSTFGQGTKVEIKRTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 7 - protein, 595 aa, synthetic construct
MALPVTALLLPLALLLHAARPAEVQLLESGGGLVQPGGSLRLSCAASGFTFSSYAMSWVRQAPGKGLEWVSSIGQKGGGTEYADSVKGRFTISRDNSKNTLYLQMNSLRAEDTAVYYCAKHFENFDYWGQGTLVTVSSGGGGSGGGGSGGGGSTDIQMTQSPSSLSASVGDRVTITCRASQSISSYLNWYQQKPGKAPKLLIYAASHLQSGVPSRFSGSGSGTDFTLTISSLQPEDFATYYCQQTRRPPSTFGQGTKVEIKRTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 8 - protein, 590 aa, synthetic construct
MALPVTALLLPLALLLHAARPAEVQLVESGGGVVQPGRSLRLSCAASGFTFSSYGMHWVRQAPGKGLEWVAVISYDGSNKYYADSVKGRFTISRDNSKNTLYLQVNSLRAEDTAVYYCARINAKWGQGTLVTVSSGGGGSGGGGSGGSALDIQMTQSPSSLSASVGDRVTITCRASQSISSYLNWYQQKPGKAPKLLIYAASSLQSGVPSRFSGSGSGTDFTLTISSLQPGDFATYYCQQSYSTPTFGQGTKVEIKRTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 9 - protein, 607 aa, synthetic construct
MALPVTALLLPLALLLHAARPQVQLVQSGAEVKKPGASVKVSCKASGYTFTNYAMHWVRQAPGQRLEWMGWINAGNGKRKYSQKFQDRVTINRDTSASTIYMELSSLGSEDTAVYYCAREEDHAGSGSYLSMDVWGQGSTVTVSSAGGGGSGGGGSGGGGSDIVMTQSPDSLAVSLGERATINCKSSQNVLYSSNNKNYLAWYQQKPGHPPKLLIYWASTRESGVPDRFSGSGSGTDFTLTITSLQTEDVAVYYCQQYYSSPLTFGGGTKVEIKTTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 10 - protein, 896 aa, synthetic construct
MALPVTALLLPLALLLHAARPSLAGGSPGAAEAPGSAQVAGLCGRLTLHRDLRTGRWEPDPQRSRRCLRDPQRVLEYCRQMYPELQIARVEQATQAIPMERWCGGSRSGSCAHPHHQVVPFRCLPGEFVSEALLVPEGCRFLHQERMDQCESSTRRHQEAQEACSSQGLILHGSGMLLPCGSDRFRGVEYVCCPPPGTPDPSGTAVGDPSTRSWPPGSRVEGAEDEEEEESFPQPVDDYFVEPPQAEEEEETVPPPSSHTLAVVGKVTPTPRPTDGVDIYFGMPGEISEHEGFLRAKMDLEERRMRQINEVMREWAMADNQSKNLPKADRQALNEHFQSILQTLEEQVSGERQRLVETHATRVIALINDQRRAALEGFLAALQADPPQAERVLLALRRYLRAEQKEQRHTLRHYQHVAAVDPEKAQQMRFQVHTHLQVIEERVNQSLGLLDQNPHLAQELRPQIQELLHSEHLGPSELEAPAPGGSSEDKGGLQPPDSKDDTPMTLPKGSTEQDAASPEKEKMNPLEQYERKVNASVPRGFPFHSSEIQRDELAPAGTGVSRETTTPAPRPPTPAPTIASQPLSLRPEACRPAAGGAVHTRGLDFACDIYIWAPLAGTCGVLLLSLVITLYCTSYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 11 - protein, 262 aa, synthetic construct
MALPVTALLLPLALLLHAARPEVQLVESGGGLVKPGGSRKLSCAASGFTFSDHGMHWVRQAPEKGLEWVAYISSGSSTIYYADTVKGRFTISRDNAKNTLFLQMTSLRSEDTAMYYCARSYIPYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 12 - protein, 257 aa, synthetic construct
MALPVTALLLPLALLLHAARPEVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSGGGGSGGGGSGGGGSSYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLS

SEQ ID NO: 13 - protein, 263 aa, synthetic construct
MALPVTALLLPLALLLHAARPEVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSGGGGSGGGGSGGGGSDIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEI

SEQ ID NO: 14 - protein, 449 aa, synthetic construct
MALPVTALLLPLALLLHAARPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 15 - protein, 367 aa, synthetic construct
MALPVTALLLPLALLLHAARPASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTT

SEQ ID NO: 16 - protein, 275 aa, synthetic construct
MALPVTALLLPLALLLHAARPAEVQLLESGGGLVQPGGSLRLSCAASGFTFSSYAMSWVRQAPGKGLEWVSNISSAGKGLEWVSSIDDSGASTYYADSVKGRFTISRDNSKNTLYLQMNSLRAEDTAVYYCAKDSASFDYWGQGTLVTVSSGGGGSGGGGSGGGGSTDIQMTQSPSSLSASVGDRVTITCRASQSISSYLNWYQQKPGKAPKLLIYTASSLQSGVPSRFSGSGSGTDFTLTISSLQPEDFATYYCQQSAASPSTFGQGTKVEIKR

SEQ ID NO: 17 - protein, 262 aa, synthetic construct
MALPVTALLLPLALLLHAARPAEVQLLESGGGLVQPGGSLRLSCAASGFTFSSYAMSWVRQAPGKGLEWVSSIGQKGGGTEYADSVKGRFTISRDNSKNTLYLQMNSLRAEDTAVYYCAKHFENFDYWGQGTLVTVSSGGGGSGGGGSGGGGSTDIQMTQSPSSLSASVGDRVTITCRASQSISSYLNWYQQKPGKAPKLLIYAASHLQSGVPSRFSGSGSGTDFTLTISSLQPEDFATYYCQQTRRPPSTFGQGTKVEIKR

SEQ ID NO: 18 - protein, 257 aa, synthetic construct
MALPVTALLLPLALLLHAARPAEVQLVESGGGVVQPGRSLRLSCAASGFTFSSYGMHWVRQAPGKGLEWVAVISYDGSNKYYADSVKGRFTISRDNSKNTLYLQVNSLRAEDTAVYYCARINAKWGQGTLVTVSSGGGGSGGGGSGGSALDIQMTQSPSSLSASVGDRVTITCRASQSISSYLNWYQQKPGKAPKLLIYAASSLQSGVPSRFSGSGSGTDFTLTISSLQPGDFATYYCQQSYSTPTFGQGTKVEIKR

SEQ ID NO: 19 - protein, 274 aa, synthetic construct
MALPVTALLLPLALLLHAARPQVQLVQSGAEVKKPGASVKVSCKASGYTFTNYAMHWVRQAPGQRLEWMGWINAGNGKRKYSQKFQDRVTINRDTSASTIYMELSSLGSEDTAVYYCAREEDHAGSGSYLSMDVWGQGSTVTVSSAGGGGSGGGGSGGGGSDIVMTQSPDSLAVSLGERATINCKSSQNVLYSSNNKNYLAWYQQKPGHPPKLLIYWASTRESGVPDRFSGSGSGTDFTLTITSLQTEDVAVYYCQQYYSSPLTFGGGTKVEIK

SEQ ID NO: 20 - protein, 563 aa, synthetic construct
MALPVTALLLPLALLLHAARPSLAGGSPGAAEAPGSAQVAGLCGRLTLHRDLRTGRWEPDPQRSRRCLRDPQRVLEYCRQMYPELQIARVEQATQAIPMERWCGGSRSGSCAHPHHQVVPFRCLPGEFVSEALLVPEGCRFLHQERMDQCESSTRRHQEAQEACSSQGLILHGSGMLLPCGSDRFRGVEYVCCPPPGTPDPSGTAVGDPSTRSWPPGSRVEGAEDEEEEESFPQPVDDYFVEPPQAEEEEETVPPPSSHTLAVVGKVTPTPRPTDGVDIYFGMPGEISEHEGFLRAKMDLEERRMRQINEVMREWAMADNQSKNLPKADRQALNEHFQSILQTLEEQVSGERQRLVETHATRVIALINDQRRAALEGFLAALQADPPQAERVLLALRRYLRAEQKEQRHTLRHYQHVAAVDPEKAQQMRFQVHTHLQVIEERVNQSLGLLDQNPHLAQELRPQIQELLHSEHLGPSELEAPAPGGSSEDKGGLQPPDSKDDTPMTLPKGSTEQDAASPEKEKMNPLEQYERKVNASVPRGFPFHSSEIQRDELAPAGTGVSRE

(19) United States
(12) Patent Application Publication
(10) Pub. No.: US 2026/0335372 A1
(43) Pub. Date: Oct. 29, 2026

(54) RECOMBINANT GLIAL CELLS COMPRISING CHIMERIC ANTIGEN RECEPTOR AND USES THEREOF

(71) Applicant: KOREA UNIVERSITY RESEARCH AND BUSINESS FOUNDATION, Seoul (KR)

(72) Inventors: Dae Sung KIM, Seoul (KR); Joon Beom BAE, Goyang-si (KR); Gyu Bum YEON, Seoul (KR); Jae Wook LEE, Namyangju-si (KR); Hyun Seo AN, Incheon (KR)

(73) Assignee: KOREA UNIVERSITY RESEARCH AND BUSINESS FOUNDATION, Seoul (KR)

(21) Appl. No.: 19/437,540

(22) Filed: Dec. 31, 2025

(30) Foreign Application Priority Data
Apr. 17, 2025 (KR) 10-2025-0050376

(51) Int. Cl. A61K 35/30; A61P 25/16; C07K 14/705; C12N 5/079; C12N 5/10

(52) U.S. Cl. CPC A61K 35/30; A61P 25/16; C07K 14/70517; C12N 5/0622; C12N 5/10; C07K 2319/03; C12N 2510/00

(57) ABSTRACT

The present invention relates to a recombinant glial cell, and more particularly, to a recombinant glial cell including a chimeric antigen receptor comprising any one of the amino acid sequences of SEQ ID NOs: 1 to 10, which can specifically recognize and bind to alpha-synuclein aggregates and induce the removal thereof.

### CROSS-REFERENCE TO RELATED APPLICATIONS

This application claims priority based on Korean Patent Application No. 10-2025-0050376 filed Apr. 17, 2025, the entire contents of which are hereby incorporated by reference.

### INCORPORATION BY REFERENCE OF SEQUENCE LISTING

The instant application contains a Sequence Listing which has been filed electronically in xml format and is hereby incorporated by reference in its entirety. Said xml file, created on Dec. 30, 2025, is named Q316667SEQLIST. xml and is 28,085 bytes in size.

### TECHNICAL FIELD

The present invention relates to a recombinant glial cell including a chimeric antigen receptor and the use thereof.

### BACKGROUND ART

Parkinson's disease (PD) is the second most prevalent neurodegenerative disease, characterized by motor dysfunction resulting from the selective degeneration of dopaminergic neurons in the substantia nigra (SNc) of the midbrain. Current treatments, including dopamine precursors such as L-DOPA, dopamine agonists, and MAO inhibitors, can improve symptoms by restoring motor function. However, the efficacy of these drugs is limited due to disease progression and the development of drug resistance upon long-term administration. Moreover, these drugs fail to address the fundamental cause of the disease and thus cannot delay disease progression. A key pathological feature of Parkinson's disease is the extracellular accumulation of the protein alpha-synuclein (αSyn), which is known to promote neuronal cell death. Although numerous treatments targeting alpha-synuclein have been developed, limitations such as difficulty in penetrating the blood-brain barrier and the potential for inducing immune responses necessitate new treatment strategies.

Astrocytes and microglia are the primary glial cells responsible for maintaining homeostasis and providing neuroprotection in the central nervous system. These glial cells are capable of removing harmful substances through phagocytosis, and may be utilized for the clearance of αSyn aggregates. However, under physiological conditions, their ability to specifically recognize and remove αSyn aggregates is limited.

Chimeric antigen receptors (CARs) are engineered receptors composed of a single-chain variable fragment (scFv) that recognizes a specific antigen fused to an intracellular signaling domain, thereby enabling targeted recognition and elimination of the antigen. When expressed in glial cells, CARs can confer the ability to specifically recognize and remove αSyn aggregates. Such engineered receptors can overcome the limitations of existing treatments and serve as an innovative therapeutic strategy for directly removing αSyn within the central nervous system.

The inventors of the present invention have developed a chimeric antigen receptor comprising an extracellular domain that specifically binds to αSyn aggregates and a phagocytosis-related intracellular signaling domain. By expressing this receptor in astrocytes, the inventors have completed the present invention.

### SUMMARY OF INVENTION

### Problems to be Solved by Invention

An object of the present invention is to provide a recombinant glial cell including a chimeric antigen receptor.

Another object of the present invention is to provide a pharmaceutical composition including the recombinant glial cell for treatment or prevention of neurodegenerative diseases.

### Means for Solving Problems

1. A recombinant glial cell including a chimeric antigen receptor comprising any one of the amino acid sequences of SEQ ID NOs: 1 to 10.
2. The recombinant glial cell according to the above 1, wherein the glial cell is an astrocyte or a microglial cell.
3. The recombinant glial cell according to the above 1, wherein the recombinant glial cell includes a chimeric antigen receptor composed of any one of the amino acid sequences of SEQ ID NOs: 2 to 4.
4. The recombinant glial cell according to the above 1, wherein the chimeric antigen receptor is introduced into the cell at a neural progenitor cell stage.
5. The recombinant glial cell according to the above 1, wherein the chimeric antigen receptor includes a CD8 transmembrane domain.
6. The recombinant glial cell according to the above 1, wherein the chimeric antigen receptor includes a MEGF10 intracellular domain.
7. The recombinant glial cell according to the above 1, wherein the chimeric antigen receptor is an alpha-synuclein aggregate-specific binding receptor.
8. A pharmaceutical composition for treating or preventing a neurodegenerative disease, including the recombinant glial cell of any one of the above 1 to 7.
9. The pharmaceutical composition according to the above 8, wherein the neurodegenerative disease is Parkinson's disease.

### Advantageous Effects

Recombinant glial cells expressing the chimeric antigen receptor of the present invention can specifically recognize and bind to alpha-synuclein aggregates.

Recombinant glial cells expressing the chimeric antigen receptor of the present invention can recognize alpha-synuclein aggregates as antigens and induce their removal through phagocytosis.

The pharmaceutical composition of the present invention can prevent or treat neurodegenerative diseases by removing accumulated alpha-synuclein aggregates or inhibiting the accumulation thereof.

### BRIEF DESCRIPTION OF DRAWINGS

FIG. 1 is a schematic diagram illustrating the gene structure of an αSyn aggregate-specific chimeric antigen receptor.

FIG. 2 illustrates the results of analyzing the αSyn aggregate clearance ability of a chimeric antigen receptor expressed in an astrocyte cell line using a pH-sensitive fluorescent probe.

FIG. 3 illustrates the results of analyzing the αSyn aggregate clearance ability of a chimeric antigen receptor expressed in a microglial cell line using a pH-sensitive fluorescent probe.

FIG. 4 illustrates the results of analyzing the αSyn aggregate degradation ability of a chimeric antigen receptor-expressing cell line.

FIG. 5 illustrates the results of comparing the expression efficiency according to the stage of astrocyte differentiation and the timing of chimeric antigen receptor introduction.

FIG. 6 illustrates the results of examining the intracellular changes in αSyn aggregates in astrocytes differentiated from human pluripotent stem cells (hPSCs).

FIG. 7 illustrates the results of confirming the inhibitory effect of chimeric antigen receptor-expressing astrocytes on neuronal damage.

FIG. 8 illustrates the results of examining the reduction effect of chimeric antigen receptor-expressing astrocytes on alpha-synuclein aggregates.

### MODE FOR CARRYING OUT INVENTION

The present invention provides a recombinant glial cell comprising a chimeric antigen receptor.

The present invention relates to a recombinant glial cell comprising a chimeric antigen receptor, and more particularly, to a recombinant glial cell expressing a chimeric antigen receptor comprising any one of the amino acid sequences of SEQ ID NOs: 1 to 10, which can specifically recognize and bind to alpha-synuclein aggregates and induce the removal thereof.

The present invention provides a recombinant glial cell comprising a chimeric antigen receptor, wherein the chimeric antigen receptor comprises any one of the amino acid sequences of SEQ ID NOs: 1 to 10.

The term “chimeric antigen receptor (CAR)” as used herein refers to a receptor that includes a single-chain antibody that recognizes a specific antigen and an intracellular signaling domain, thereby exhibiting high efficiency in specifically recognizing and inducing the removal of the target antigen.

The CAR of the present invention may include an extracellular domain (ECD), a transmembrane domain, and an intracellular signaling domain. The specific function or activity of the CAR may vary depending on the selection and/or combination of the extracellular domain, the transmembrane domain, and the intracellular signaling domain.

In addition, the CAR may include an antibody, a single-domain antibody, or a single-chain variable fragment (scFv) that specifically binds to αSyn aggregates.

Further, the CAR may further include at least one of a signal peptide, a glycine-serine (GS) linker, a transmembrane domain, and an intracellular domain, and is not limited thereto, provided that it includes components generally known for a CAR.

The extracellular domain may include any one of the amino acid sequences represented by SEQ ID NOs: 11 to 20, preferably an amino acid sequence having at least 90% sequence identity with any one of the amino acid sequences of SEQ ID NOs: 11 to 20, more preferably at least 95%, and most preferably at least 98%.

Sequence identity for the amino acid sequence is determined by comparing an optimally aligned sequence with a comparison region. In the comparison region, a portion of the amino acid sequence may include additions or deletions (i.e., gaps) of amino acids when compared with a reference sequence for the optimal alignment (excluding additions or deletions).

The single-chain variable fragment may refer to a protein that links the variable regions of the light and heavy chains of an antibody. The two variable regions are connected by a linker peptide consisting of approximately 15 amino acids, and may have a structure of a light chain variable region-linker-heavy chain variable region or a heavy chain variable region-linker-light chain variable region. Such a single-chain variable fragment retains the antigen recognition properties of the original antibody. The linker may use a flexible hydrophilic peptide chain (glycine-serine (GS) linker) mainly composed of glycine and serine, and typically has a 15-amino-acid sequence such as “(Gly-Gly-Gly-Gly-Ser) 3” or a similar structure.

The extracellular domain of the present invention has excellent binding affinity to αSyn aggregates.

The extracellular domain may include a single-chain variable fragment having high binding affinity for αSyn aggregates, and the extracellular domain may include a nucleic acid sequence encoding a receptor having high binding affinity for αSyn aggregates.

The signal peptide may include, for example, a CD8a signal peptide sequence.

The transmembrane domain may include, for example, a CD8 hinge or a CD8 transmembrane domain.

The intracellular signaling domain may include, for example, a MEGF10 intracellular domain.

The CAR comprising the MEGF10 intracellular domain may induce enhanced phagocytic activity.

The recombinant glial cells of the present invention express a CAR comprising the MEGF10 signaling sequence, which induces highly efficient phagocytosis, and thus may effectively phagocytose αSyn aggregates.

The CAR of the present invention may comprise any one of the amino acid sequences of SEQ ID NOs: 1 to 10.

The CAR comprising any one of the amino acid sequences of SEQ ID NOs: 1 to 10 may include a predetermined extracellular domain (ECD), thereby specifically recognizing and binding to αSyn aggregates. The CAR may be an αSyn aggregate-specific binding receptor.

In one embodiment, the CAR of the present invention may effectively recognize αSyn aggregates and traffic the same to lysosomes.

Therefore, recombinant glial cells comprising a CAR, wherein the CAR comprises any one of the amino acid sequences of SEQ ID NOs: 1 to 10 may exhibit enhanced αSyn aggregate-specific phagocytic activity by comprising a signaling domain that induces phagocytic activation in glial cells.

The CAR of the present invention may be expressed on the surface of phagocytic cells, recognize and bind to a target antigen, thereby inducing activation of the phagocytic cells. The receptor may be configured to selectively recognize a specific antigen present on a target cell to be eliminated, and the target cell may be a cell present in a specific tissue or a cell present at a disease-causing lesion site.

In addition, the CAR of the present invention may be composed of any one of the amino acid sequences of SEQ ID NOs: 2 to 4. The receptor may effectively recognize αSyn aggregates and traffic the same to lysosomes.

Further, the CAR comprising any one of the amino acid sequences of SEQ ID NOs: 1 to 10 of the present invention may include functionally equivalent variants. The term “functionally equivalent variant” refers to a protein generated by insertion, substitution, or deletion of one or more amino acids, and having at least 70% sequence identity to a CAR protein comprising any one of the amino acid sequences of SEQ ID NOs: 1 to 10, and may have sequence identity of 80%, 90%, or 95% or more. The variant performs essentially the same biological function as the CAR protein comprising any one of the amino acid sequences of SEQ ID NOs: 1 to 10, and specifically retains the ability to selectively recognize and bind to αSyn aggregates.

Further, the present invention includes fragments, variants, and analog molecules of the CAR.

The terms “fragment,” “variant,” and “analog molecule” may refer to polypeptides that retain the essential biological function and activity of the CAR of the present invention, and the fragments, variants, and analog molecules may exist in the following forms: (1) a polypeptide in which one or more amino acids are substituted with another amino acid, preferably with an amino acid having similar properties, and wherein the amino acid may be naturally occurring or non-naturally occurring; (2) a polypeptide in which one or more amino acid residues are chemically modified; (3) a polypeptide to which a substance that increases the stability of the polypeptide (e.g., a substance that extends the half-life, such as polyethylene glycol) is bound; and (4) a polypeptide to which an additional amino acid sequence for a specific purpose is linked, wherein such additional sequences may include a signal peptide, a secretory sequence, a purification tag sequence, a precursor protein sequence, or a sequence for a fusion protein.

The term “glial cells (neuroglia)” as used herein refers to cells that protect, assist, or support nerve cells (neurons) in the nervous system, and may be used interchangeably with the term “glia.”

The glial cell may be an astrocyte, a microglial cell, an ependymocyte, or an oligodendrocyte, and is preferably an astrocyte or a microglial cell.

The recombinant glial cells of the present invention may be differentiated into glial cells from neural progenitor cells into which the CAR has been introduced.

The CAR may be introduced at the neural progenitor cell stage.

In one embodiment, the cells may be obtained or prepared from hPSCs.

In one embodiment, when the CAR gene of the present invention was introduced into neural progenitor cells differentiated from human pluripotent stem cells and subsequently differentiated into astrocytes, the CAR was most efficiently expressed.

The term “recombinant glial cells” of the present invention may refer to glial cells into which a CAR comprising any one of the amino acid sequences of SEQ ID NOs: 1 to 10 has been introduced and expressed, but is not limited thereto.

The term “introduction” may refer to transfection or transduction, and may, for example, include transforming glial cells with a recombinant vector.

The recombinant vector may be configured by appropriately selecting and combining expression regulatory sequences, such as a promoter, a terminator, and an enhancer, and a signal sequence for membrane targeting or secretion of a protein, depending on the characteristics of a host cell for expression of the CAR. The vector may include, but is not limited to, plasmid vectors, cosmid vectors, bacteriophage vectors, and viral vectors. The vector may include regulatory elements, such as a promoter, an operator, an initiation codon, a termination codon, a polyadenylation signal, and an enhancer, for regulating gene expression. In addition, the vector may be variously prepared to include a signal sequence or a leader sequence for membrane targeting or secretion of a protein, depending on the intended purpose.

The recombinant vector may be a viral vector comprising a gene encoding a CAR, and the viral vector may be, for example, a lentivirus, a retrovirus, an adenovirus, an adeno-associated virus, a Sendai virus, an episomal vector, or a BAC vector.

Glial cells into which the CAR of the present invention has been introduced may recognize αSyn aggregates as antigens and bind strongly thereto. By such introduction, the glial cells can specifically respond to αSyn aggregates, rather than to receptors that are naturally phagocytosed by the glial cells prior to the introduction.

Glial cells transduced with the CAR of the present invention may induce the removal of αSyn aggregates through phagocytosis.

The CAR of the present invention may be introduced into phagocytic cells, and may, for example, be expressed in the glial cells.

The CAR of the present invention may be expressed in the glial cells to promote phagocytosis of αSyn aggregates.

Furthermore, the present invention includes a pharmaceutical composition for treatment or prevention of neurodegenerative diseases, comprising the recombinant glial cell.

The term “neurodegenerative disease” refers to a disease in which cells of the central nervous system cease functioning or die due to structural alterations in specific regions of the brain, and includes, for example, Alzheimer's disease, Parkinson's disease, Huntington's disease, or amyotrophic lateral sclerosis.

The term “treatment” refers to any action by which symptoms of an αSyn aggregate-related disease are ameliorated or beneficially altered by administration of a pharmaceutical composition comprising the recombinant glial cell of the present invention.

The term “prevention” refers to any action that inhibits the occurrence of or delays the onset of an αSyn aggregate-related disease by administration of a pharmaceutical composition comprising the recombinant glial cell of the present invention.

As used herein, the term “pharmaceutical composition” may be in the form of capsules, tablets, granules, injections, ointments, powders, or beverages, and the pharmaceutical composition may be administered to humans. The pharmaceutical composition is not limited to these forms, but may be formulated according to conventional methods into oral dosage forms such as powders, granules, capsules, tablets, and aqueous suspensions, as well as topical preparations, suppositories, and sterile injectable solutions.

The pharmaceutical composition of the present invention may include a pharmaceutically acceptable carrier. Pharmaceutically acceptable carriers usable for oral administration may include binders, lubricants, disintegrants, excipients, solubilizers, dispersants, stabilizers, suspending agents, coloring agents, and flavoring agents. For injections, buffers, preservatives, analgesic agents, solubilizers, isotonic agents, and stabilizers may be mixed and used. For topical administration, bases, excipients, lubricants, and preservatives may be used.

The pharmaceutical composition of the present invention may be prepared in various forms by mixing it with the pharmaceutically acceptable carriers described above. For example, for oral administration, the pharmaceutical composition may be prepared in the form of tablets, troches, capsules, elixirs, suspensions, syrups, or wafers, and for injectable formulations, it may be prepared in the form of unit-dose ampoules or multi-dose preparations. In addition, it may be formulated as solutions, suspensions, tablets, capsules, or sustained-release preparations.

Meanwhile, examples of carriers, excipients, and diluents suitable for formulation may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, or mineral oil, and may further include fillers, anti-caking agents, lubricants, wetting agents, flavoring agents, emulsifiers, preservatives, and the like.

The route of administration of the pharmaceutical composition according to the present invention may include, but is not limited to, oral, intravenous, intramuscular, intraarterial, intramedullary, intrathecal, intracardiac, transdermal, subcutaneous, intraperitoneal, intranasal, enteral, topical, sublingual, or rectal, with oral or parenteral administration being preferred. The term “parenteral” as used herein includes subcutaneous, intradermal, intravenous, intramuscular, intraarticular, intrasynovial, intrasternal, intrathecal, intralesional, and intracranial injection or infusion techniques. The pharmaceutical composition of the present invention may also be administered in the form of a suppository for rectal administration.

The formulation and administration of the pharmaceutical composition of the present invention may vary depending on various factors, including the activity of the specific compound used, age, body weight, general health, gender, dietary status, time of administration, route of administration, excretion rate, drug combination, and the severity of the disease associated with αSyn aggregates to be prevented or treated. The dosage of the pharmaceutical composition may vary depending on the patient's condition, body weight, severity of the disease, dosage form, route of administration, and duration, but may be appropriately selected by those skilled in the art. The dosage may be administered in an amount of 0.0001 to 500 mg/kg or 0.001 to 500 mg/kg per day. The dosage may be administered once daily or divided into multiple doses. Such dosage is not intended to limit the scope of the present invention in any manner. The pharmaceutical composition according to the present invention may be formulated as a pill, sugar-coated tablet, capsule, solution, gel, syrup, slurry, or suspension.

Hereinafter, the present invention will be described in more detail by way of examples.

### EXAMPLES

### Example 1. Preparation of a CAR Expression Vector That Specifically Binds to αSyn Aggregates

To prepare the CAR of the present invention, coding sequence for the extracellular domains of ten CARs, each comprising a CD8a signal peptide and either seven different single-chain variable fragments or three different receptor extracellular domains were synthesized. The coding sequence for the CD8a transmembrane domain and MEGF10 intracellular signaling domain were then inserted into the synthesized sequences to generate an αSyn aggregate-specific CAR expression vector.

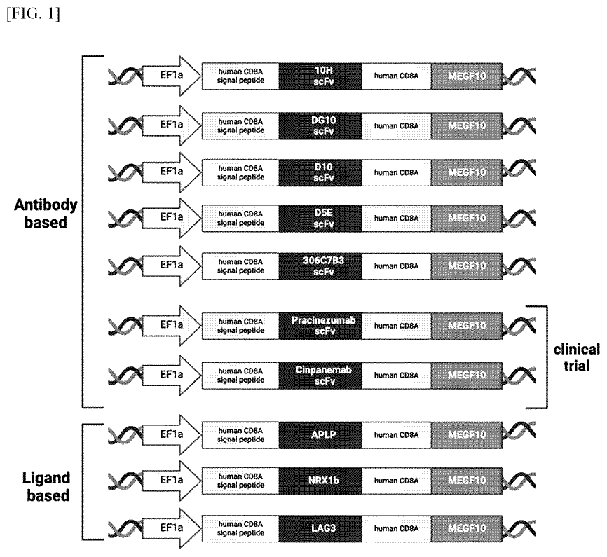

The gene and protein sequence combinations of each vector thus prepared are shown in Table 1, and a schematic diagram of the gene arrangement is shown in FIG. 1. The corresponding amino acid sequences are shown in Table 2.

TABLE 1

| Sequence No. | Gene arrangement |
|---|---|
| SEQ ID NO. 1 | 306C7B3-hMEGF10 |
| SEQ ID NO. 2 | Cinpanemab_scFv-hMEGF10 |
| SEQ ID NO. 3 | Prasinezumab_scFv-hMEGF10 |
| SEQ ID NO. 4 | LAG3-hMEGF10 |
| SEQ ID NO. 5 | NRX1B-hMEGF10 |
| SEQ ID NO. 6 | 10H-hMEGF10 |
| SEQ ID NO. 7 | D5E-hMEGF10 |
| SEQ ID NO. 8 | D10-hMEGF10 |
| SEQ ID NO. 9 | DG10-hMEGF10 |
| SEQ ID NO. 10 | APLP1-hMEGF10 |

TABLE 2

| Sequence No. | Amino acid sequences |
|---|---|
| SEQ ID NO. 1 | MALPVTALLLPLALLLHAARPEVQLVESGGGLVKP |
| | GGSRKLSCAASGFTFSDHGMHWVRQAPEKGLEWVA |
| | YISSGSSTIYYADTVKGRFTISRDNAKNTLFLQMT |
| | SLRSEDTAMYYCARSYIPYWGQGTLVTVSAGGGGS |
| | GGGGSGGGGSDVVMTQTPLSLPVSLGDQASISCRS |
| | SQSLVHSNGNTYLHWYLQKPGQSPKLLIYKVSNRF |
| | SGVPDRFSGSGSGTDFTLKISRVEAEDLGVYFCSQ |
| | STHVPWTFGGGTKLEIKTTTPAPRPPTPAPTIASQ |
| | PLSLRPEACRPAAGGAVHTRGLDFACDIYIWAPLA |
| | GTCGVLLLSLVITLYCTSYRHKQKGKESSMPAVTY |
| | TPAMRVVNADYTISGTLPHSNGGNANSHYFTNPSY |
| | HTLTQCATSPHVNNRDRMTVTKSKNNQLFVNLKNV |
| | NPGKRGPVGDCTGTLPADWKHGGYLNELGAFGLDR |
| | SYMGKSLKDLGKNSEYNSSNCSLSSSENPYATIKD |
| | PPVLIPKSSECGYVEMKSPARRDSPYAEINNSTSA |
| | NRNVYEVEPTVSVVQGVFSNNGRLSQDPYDLPKNS |
| | HIPCHYDLLPVRDSSSSPKQEDSGGSSSNSSSSSE |
| SEQ ID NO. 2 | MALPVTALLLPLALLLHAARPEVQLVESGGGLVEP |
| | GGSLRLSCAVSGFDFEKAWMSWVRQAPGQGLQWVA |
| | RIKSTADGGTTSYAAPVEGRFIISRDDSRNMLYLQ |
| | MNSLKTEDTAVYYCTSAHWGQGTLVTVSGGGGSGG |
| | GGSGGGGSSYELTQPPSVSVSPGQTARITCSGEAL |
| | PMQFAHWYQQRPGKAPVIVVYKDSERPSGVPERFS |
| | GSSSGTTATLTITGVQAEDEADYYCQSPDSTNTYE |
| | VFGGGTKLTVLSTTTPAPRPPTPAPTIASQPLSLR |
| | PEACRPAAGGAVHTRGLDFACDIYIWAPLAGTCGV |
| | LLLSLVITLYCTSYRHKQKGKESSMPAVTYTPAMR |
| | VVNADYTISGTLPHSNGGNANSHYFTNPSYHTLTQ |
| | CATSPHVNNRDRMTVTKSKNNQLFVNLKNVNPGKR |
| | GPVGDCTGTLPADWKHGGYLNELGAFGLDRSYMGK |
| | SLKDLGKNSEYNSSNCSLSSSENPYATIKDPPVLI |
| | PKSSECGYVEMKSPARRDSPYAEINNSTSANRNVY |
| | EVEPTVSVVQGVFSNNGRLSQDPYDLPKNSHIPCH |
| | YDLLPVRDSSSSPKQEDSGGSSSNSSSSSE |
| SEQ ID NO. 3 | MALPVTALLLPLALLLHAARPEVQLVESGGGLVQP |
| | GGSLRLSCAASGFTFSNYGMSWVRQAPGKGLEWVA |
| | SISSGGGSTYYPDNVKGRFTISRDDAKNSLYLQMN |
| | SLRAEDTAVYYCARGGAGIDYWGQGTLVTVSGGGG |
| | SGGGGSGGGGSDIQMTQSPSSLSASVGDRVTITCK |
| | SIQTLLYSSNQKNYLAWFQQKPGKAPKLLIYWASI |
| | RKSGVPSRFSGSGSGTDFTLTISSLQPEDLATYYC |
| | QQYYSYPLTFGGGTKLEITTTPAPRPPTPAPTIAS |
| | QPLSLRPEACRPAAGGAVHTRGLDFACDIYIWAPL |
| | AGTCGVLLLSLVITLYCTSYRHKQKGKESSMPAVT |
| | YTPAMRVVNADYTISGTLPHSNGGNANSHYFTNPS |
| | YHTLTQCATSPHVNNRDRMTVTKSKNNQLFVNLKN |
| | VNPGKRGPVGDCTGTLPADWKHGGYLNELGAFGLD |
| | RSYMGKSLKDLGKNSEYNSSNCSLSSSENPYATIK |
| | DPPVLIPKSSECGYVEMKSPARRDSPYAEINNSTS |
| | ANRNVYEVEPTVSVVQGVFSNNGRLSQDPYDLPKN |
| | SHIPCHYDLLPVRDSSSSPKQEDSGGSSSNSSSSS |
| | E |
| SEQ ID NO. 4 | MALPVTALLLPLALLLHAARPLQPGAEVPVVWAQE |
| | GAPAQLPCSPTIPLQDLSLLRRAGVTWQHQPDSGP |
| | PAAAPGHPLAPGPHPAAPSSWGPRPRRYTVLSVGP |
| | GGLRSGRLPLQPRVQLDERGRQRGDFSLWLRPARR |
| | ADAGEYRAAVHLRDRALSCRLRLRLGQASMTASPP |
| | GSLRASDWVILNCSFSRPDRPASVHWFRNRGQGRV |
| | PVRESPHHHLAESFLFLPQVSPMDSGPWGCILTYR |
| | DGFNVSIMYNLTVLGLEPPTPLTVYAGAGSRVGLP |
| | CRLPAGVGTRSFLTAKWTPPGGGPDLLVTGDNGDF |
| | TLRLEDVSQAQAGTYTCHIHLQEQQLNATVTLAII |
| | TVTPKSFGSPGSLGKLLCEVTPVSGQERFVWSSLD |
| | TPSQRSFSGPWLEAQEAQLLSQPWQCQLYQGERLL |
| | GAAVYFTELSSPGAQRSGRAPGALPAGHLTTTPAP |
| | RPPTPAPTIASQPLSLRPEACRPAAGGAVHTRGLD |
| | FACDIYIWAPLAGTCGVLLLSLVITLYCTSYRHKQ |
| | KGKESSMPAVTYTPAMRVVNADYTISGTLPHSNGG |
| | NANSHYFTNPSYHTLTQCATSPHVNNRDRMTVTKS |
| | KNNQLFVNLKNVNPGKRGPVGDCTGTLPADWKHGG |
| | YLNELGAFGLDRSYMGKSLKDLGKNSEYNSSNCSL |
| | SSSENPYATIKDPPVLIPKSSECGYVEMKSPARRD |
| | SPYAEINNSTSANRNVYEVEPTVSVVQGVFSNNGR |
| | LSQDPYDLPKNSHIPCHYDLLPVRDSSSSPKQEDS |
| | GGSSSNSSSSSE |
| SEQ ID NO. 5 | MALPVTALLLPLALLLHAARPASSLGAHHIHHFHG |
| | SSKHHSVPIAIYRSPASLRGGHAGTTYIFSKGGGQ |
| | ITYKWPPNDRPSTRADRLAIGFSTVQKEAVLVRVD |
| | SSSGLGDYLELHIHQGKIGVKFNVGTDDIAIEESN |
| | AIINDGKYHVVRFTRSGGNATLQVDSWPVIERYPA |
| | GNNDNERLAIARQRIPYRLGRVVDEWLLDKGRQLT |
| | IFNSQATIIIGGKEQGQPFQGQLSGLYYNGLKVLN |
| | MAAENDANIAIVGNVRLVGEVPSSMTTESTATAMQ |
| | SEMSTSIMETTTTLATSTARRGKPPTKEPISQTTD |
| | DILVASAECPSDDEDIDPCEPSSGGLANPTRAGGR |
| | EPYPGSAEVIRESSSTTTTTPAPRPPTPAPTIASQ |
| | PLSLRPEACRPAAGGAVHTRGLDFACDIYIWAPLA |
| | GTCGVLLLSLVITLYCTSYRHKQKGKESSMPAVTY |
| | TPAMRVVNADYTISGTLPHSNGGNANSHYFTNPSY |
| | HTLTQCATSPHVNNRDRMTVTKSKNNQLFVNLKNV |
| | NPGKRGPVGDCTGTLPADWKHGGYLNELGAFGLDR |
| | SYMGKSLKDLGKNSEYNSSNCSLSSSENPYATIKD |
| | PPVLIPKSSECGYVEMKSPARRDSPYAEINNSTSA |
| | NRNVYEVEPTVSVVQGVFSNNGRLSQDPYDLPKNS |
| | HIPCHYDLLPVRDSSSSPKQEDSGGSSSNSSSSSE |
| SEQ ID NO. 6 | MALPVTALLLPLALLLHAARPAEVQLLESGGGLVQ |
| | PGGSLRLSCAASGFTFSSYAMSWVRQAPGKGLEWV |
| | SNISSAGKGLEWVSSIDDSGASTYYADSVKGRFTI |
| | SRDNSKNTLYLQMNSLRAEDTAVYYCAKDSASFDY |
| | WGQGTLVTVSSGGGGSGGGGSGGGGSTDIQMTQSP |
| | SSLSASVGDRVTITCRASQSISSYLNWYQQKPGKA |
| | PKLLIYTASSLQSGVPSRFSGSGSGTDFTLTISSL |
| | QPEDFATYYCQQSAASPSTFGQGTKVEIKRTTTPA |
| | PRPPTPAPTIASQPLSLRPEACRPAAGGAVHTRGL |
| | DFACDIYIWAPLAGTCGVLLLSLVITLYCTSYRHK |
| | QKGKESSMPAVTYTPAMRVVNADYTISGTLPHSNG |
| | GNANSHYFTNPSYHTLTQCATSPHVNNRDRMTVTK |
| | SKNNQLFVNLKNVNPGKRGPVGDCTGTLPADWKHG |
| | GYLNELGAFGLDRSYMGKSLKDLGKNSEYNSSNCS |
| | LSSSENPYATIKDPPVLIPKSSECGYVEMKSPARR |
| | DSPYAEINNSTSANRNVYEVEPTVSVVQGVFSNNG |
| | RLSQDPYDLPKNSHIPCHYDLLPVRDSSSSPKQED |
| | SGGSSSNSSSSSE |
| SEQ ID NO. 7 | MALPVTALLLPLALLLHAARPAEVQLLESGGGLVQ |
| | PGGSLRLSCAASGFTFSSYAMSWVRQAPGKGLEWV |
| | SSIGQKGGGTEYADSVKGRFTISRDNSKNTLYLQM |
| | NSLRAEDTAVYYCAKHFENFDYWGQGTLVTVSSGG |
| | GGSGGGGSGGGGSTDIQMTQSPSSLSASVGDRVTI |
| | TCRASQSISSYLNWYQQKPGKAPKLLIYAASHLQS |
| | GVPSRFSGSGSGTDFTLTISSLQPEDFATYYCQQT |
| | RRPPSTFGQGTKVEIKRTTTPAPRPPTPAPTIASQ |
| | PLSLRPEACRPAAGGAVHTRGLDFACDIYIWAPLA |
| | GTCGVLLLSLVITLYCTSYRHKQKGKESSMPAVTY |
| | TPAMRVVNADYTISGTLPHSNGGNANSHYFTNPSY |
| | HTLTQCATSPHVNNRDRMTVTKSKNNQLFVNLKNV |
| | NPGKRGPVGDCTGTLPADWKHGGYLNELGAFGLDR |
| | SYMGKSLKDLGKNSEYNSSNCSLSSSENPYATIKD |
| | PPVLIPKSSECGYVEMKSPARRDSPYAEINNSTSA |
| | NRNVYEVEPTVSVVQGVFSNNGRLSQDPYDLPKNS |
| | HIPCHYDLLPVRDSSSSPKQEDSGGSSSNSSSSSE |
| SEQ ID NO. 8 | MALPVTALLLPLALLLHAARPAEVQLVESGGGVVQ |
| | PGRSLRLSCAASGFTFSSYGMHWVRQAPGKGLEWV |
| | AVISYDGSNKYYADSVKGRFTISRDNSKNTLYLQV |
| | NSLRAEDTAVYYCARINAKWGQGTLVTVSSGGGGS |
| | GGGGSGGSALDIQMTQSPSSLSASVGDRVTITCRA |
| | SQSISSYLNWYQQKPGKAPKLLIYAASSLQSGVPS |
| | RFSGSGSGTDFTLTISSLQPGDFATYYCQQSYSTP |
| | TFGQGTKVEIKRTTTPAPRPPTPAPTIASQPLSLR |
| | PEACRPAAGGAVHTRGLDFACDIYIWAPLAGTCGV |
| | LLLSLVITLYCTSYRHKQKGKESSMPAVTYTPAMR |
| | VVNADYTISGTLPHSNGGNANSHYFTNPSYHTLTQ |
| | CATSPHVNNRDRMTVTKSKNNQLFVNLKNVNPGKR |
| | GPVGDCTGTLPADWKHGGYLNELGAFGLDRSYMGK |
| | SLKDLGKNSEYNSSNCSLSSSENPYATIKDPPVLI |
| | PKSSECGYVEMKSPARRDSPYAEINNSTSANRNVY |
| | EVEPTVSVVQGVFSNNGRLSQDPYDLPKNSHIPCH |
| | YDLLPVRDSSSSPKQEDSGGSSSNSSSSSE |
| SEQ ID NO. 9 | MALPVTALLLPLALLLHAARPQVQLVQSGAEVKKP |
| | GASVKVSCKASGYTFTNYAMHWVRQAPGQRLEWMG |
| | WINAGNGKRKYSQKFQDRVTINRDTSASTIYMELS |
| | SLGSEDTAVYYCAREEDHAGSGSYLSMDVWGQGST |
| | VTVSSAGGGGGGGGSGGGGSDIVMTQSPDSLAVSL |
| | GERATINCKSSQNVLYSSNNKNYLAWYQQKPGHPP |
| | KLLIYWASTRESGVPDRFSGSGSGTDFTLTITSLQ |
| | TEDVAVYYCQQYYSSPLTFGGGTKVEIKTTTPAPR |
| | PPTPAPTIASQPLSLRPEACRPAAGGAVHTRGLDF |
| | ACDIYIWAPLAGTCGVLLLSLVITLYCTSYRHKQK |
| | GKESSMPAVTYTPAMRVVNADYTISGTLPHSNGGN |
| | ANSHYFTNPSYHTLTQCATSPHVNNRDRMTVTKSK |
| | NNQLFVNLKNVNPGKRGPVGDCTGTLPADWKHGGY |
| | LNELGAFGLDRSYMGKSLKDLGKNSEYNSSNCSLS |
| | SSENPYATIKDPPVLIPKSSECGYVEMKSPARRDS |
| | PYAEINNSTSANRNVYEVEPTVSVVQGVFSNNGRL |
| | SQDPYDLPKNSHIPCHYDLLPVRDSSSSPKQEDSG |
| | GSSSNSSSSSE |
| SEQ ID NO. 10 | MALPVTALLLPLALLLHAARPSLAGGSPGAAEAPG |
| | SAQVAGLCGRLTLHRDLRTGRWEPDPQRSRRCLRD |
| | PQRVLEYCRQMYPELQIARVEQATQAIPMERWCGG |
| | SRSGSCAHPHHQVVPFRCLPGEFVSEALLVPEGCR |
| | FLHQERMDQCESSTRRHQEAQEACSSQGLILHGSG |
| | MLLPCGSDRFRGVEYVCCPPPGTPDPSGTAVGDPS |
| | TRSWPPGSRVEGAEDEEEEESFPQPVDDYFVEPPQ |
| | AEEEEETVPPPSSHTLAVVGKVTPTPRPTDGVDIY |
| | FGMPGEISEHEGFLRAKMDLEERRMRQINEVMREW |
| | AMADNQSKNLPKADRQALNEHFQSILQTLEEQVSG |
| | ERQRLVETHATRVIALINDQRRAALEGFLAALQAD |
| | PPQAERVLLALRRYLRAEQKEQRHTLRHYQHVAAV |
| | DPEKAQQMRFQVHTHLQVIEERVNQSLGLLDQNPH |
| | LAQELRPQIQELLHSEHLGPSELEAPAPGGSSEDK |
| | GGLQPPDSKDDTPMTLPKGSTEQDAASPEKEKMNP |
| | LEQYERKVNASVPRGFPFHSSEIQRDELAPAGTGV |
| | SRETTTPAPRPPTPAPTIASQPLSLRPEACRPAAG |
| | GAVHTRGLDFACDIYIWAPLAGTCGVLLLSLVITL |
| | YCTSYRHKQKGKESSMPAVTYTPAMRVVNADYTIS |
| | GTLPHSNGGNANSHYFTNPSYHTLTQCATSPHVNN |
| | RDRMTVTKSKNNQLFVNLKNVNPGKRGPVGDCTGT |
| | LPADWKHGGYLNELGAFGLDRSYMGKSLKDLGKNS |
| | EYNSSNCSLSSSENPYATIKDPPVLIPKSSECGYV |
| | EMKSPARRDSPYAEINNSTSANRNVYEVEPTVSVV |
| | QGVFSNNGRLSQDPYDLPKNSHIPCHYDLLPVRDS |
| | SSSPKQEDSGGSSSNSSSSSE |

### Example 2. Preparation of a Recombinant Lentivirus for Expression of a CAR That Specifically Binds to αSyn Aggregates

To prepare a recombinant lentivirus for expression of a CAR that specifically binds to αSyn aggregates, the HEK293FT cell line (Invitrogen) were prepared as follows. Complete cell culture medium was prepared by supplementing 10% fetal bovine serum (FBS) and 1× Penicillin-Streptomycin (Gibco) to DMEM (Gibco). The HEK293FT cell line was thawed in a 37° C. water bath for 2-3 minutes, resuspended into 5 mL of complete medium, and cultured at 37° C. in 5% CO₂. Cells were maintained by subculture upon reaching 80% confluency. For lentiviral transfection, the HEK293FT cell line was seeded at density 1-2×10⁶ cells in a 10 cm culture dish containing 10 mL of complete medium, and transfection was performed after culturing for 16 hours.

Lentiviral transfection was performed using Lipofectamine 3000 (Invitrogen). 4 μg of the expression vector and 1.3 μg each of the pRSV-Rev, pMDLg/pRRE, and pMD2.G packaging vectors, were diluted in 500 μL of Opti-MEM (Invitrogen) with 30 μL of P3000 reagent. Separately, 30 μL of Lipofectamine 3000 was added to 500 μL of Opti-MEM, and the two solutions were mixed and incubated at room temperature for 20 minutes. The mixture was added dropwise to the prepared HEK293FT cells and gently mixed. After 16 hours of incubation, the medium was replaced with 9 mL of fresh complete medium and incubated for an additional 32 hours.

After incubation, the supernatant was collected and stored at 4° C., and the cells were replenished with 9 mL of fresh complete medium. After 24 hours, all collected supernatants were combined and filtered through a 0.45-μm filter to remove cell debris. Lenti-X Concentrator (Takara) was added in an amount corresponding to one-third of the total supernatant volume, and the mixture was incubated overnight at 4° C. The mixture was centrifuged at 1,500×g for 45 minutes to concentrate the lentivirus. The concentrated lentivirus was resuspended in phosphate-buffered saline (PBS) and stored at −80° C. before use.

### Example 3. Preparation of Immortalized Mouse Astrocytes and HMC3 Microglial Cells Expressing a CAR That Specifically Binds to αSyn Aggregates

To prepare immortalized mouse astrocytes and HMC3 cells expressing a CAR that specifically binds to αSyn aggregates, each cell line was transduced with recombinant lentiviruses as described in Example 2. Specifically, recombinant lentiviruses at multiplicities of infection (MOIs) ranging from 0.1 to 10 were added to complete medium supplemented with 8 μg/mL polybrene (Merck). 1 mL of the mixture was added to each cell line seeded in a 6-well plate, followed by centrifugation at 1,000×g for 60 minutes and incubation at 37° C. in 5% CO₂ for 24 hours. After 24 hours, the medium was replaced with fresh medium.

To confirm the expression of a CAR that specifically binds to αSyn aggregates in transduced cell lines, cells were harvested, washed with PBS, and the expression of mRuby3 fluorescent protein, linked via a P2A sequence, was analyzed by flow cytometry.

Flow cytometric analysis confirmed that immortalized mouse astrocytes and HMC3 cells expressing the CAR were successfully prepared.

### Example 4. Screening of the αSyn Aggregate Clearance Ability of a CAR Using a pH-Sensitive Fluorescent Probe

To track the intracellular trafficking pathway of αSyn aggregates, the pH-dependent fluorescent dye Protonex Green was conjugated to αSyn aggregates. First, recombinant human αSyn protein was dissolved in DPBS at 1 mg/mL and agitated at 1,000 rpm at 37° C. conditions for 7 days using a thermomixer (Eppendorf) to prepare αSyn aggregates. The prepared αSyn aggregates were incubated with Protonex Green (AAT Bioquest), a pH-dependent fluorescent dye, at room temperature for 3 hours according to the manufacturer's protocol. After the reaction, excess fluorescent dye was removed using a 3-kDa cut-off centrifugal filter. The fluorescently labeled αSyn aggregates were sonicated using a probe sonicator (3× 10 seconds).

The ten CAR-expressing astrocyte cell lines prepared in Example 3 were seeded in 24-well plates at 1×10⁵ cells per well, and 24 hours later, were treated with Protonex Green-labeled αSyn aggregates at 1 μg/mL. Cells were harvested at 4, 8, 12, and 24 hours after treatment, washed twice with PBS, and the intracellular Protonex Green fluorescence intensity was measured by flow cytometry. Since Protonex Green exhibits increased fluorescence intensity under low-pH conditions in lysosomes, an increase in fluorescence intensity indicates the trafficking of αSyn aggregates to lysosomes.

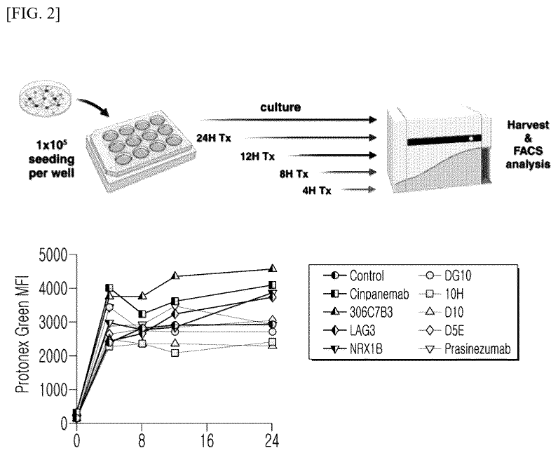

Analysis revealed that, among the ten CAR-expressing astrocyte cell lines, four cell lines expressing CAR containing the cinpanemab scFv, the 306C7B3 scFv, an extracellular domain of LAG3, and an extracellular domain of NRX1B showed a statistically significant, time-dependent increase in fluorescence intensity. Notably, the highest fluorescence intensity was observed at 24 hours post-treatment, indicating that these CARs can effectively recognize αSyn aggregates and traffic them to lysosomes (FIG. 2).

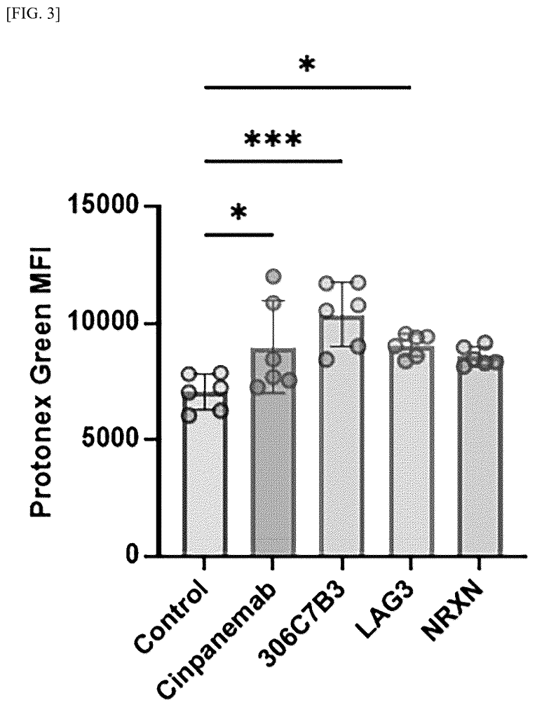

The four CAR-expressing microglial cell lines prepared in Example 3 were treated for 24 hours using the same method as described above for astrocyte cell line and then analyzed. Among the four CAR-expressing microglial cell lines, three CAR-expressing cell lines containing the cinpanemab scFv, the 306C7B3 scFv, and an extracellular domain of LAG3 exhibited uptake and lysosomal trafficking of αSyn aggregates (FIG. 3).

### Example 5. Analysis of the αSyn Aggregate Degradation Ability of CAR-Expressing Cell Lines

Western blot analysis was performed to assess the ability of cell lines to degrade αSyn aggregate expressing the CAR selected in Example 4. Each cell line was treated with αSyn aggregates at 1 μg/mL and cultured for 24 hours with or without the lysosomal inhibitor chloroquine (25 μM) treatment.

After incubation, cells were harvested and lysed in RIPA buffer (50 mM Tris-HCl, pH 7.4, 150 mM NaCl, 1% NP-40, 0.1% SDS, 0.5% sodium deoxycholate, and protease inhibitor cocktail). The soluble fraction was obtained by centrifugation at 13,000 rpm for 20 minutes. The insoluble pellet was resuspended in 2% SDS buffer (50 mM Tris-HCl, pH 7.4, 2% SDS).

Protein samples were separated on a 4-12% Bis-Tris polyacrylamide gel. The Bis-Tris gel was prepared with a thickness of 1.5 mm. The separation gel was prepared by mixing 6 mL of 3× Bis-Tris gel buffer (1 M Bis-Tris, pH 6.5-6.8), 3.6 mL of 40% acrylamide stock solution (29:1), and 8.4 mL of distilled water, to a final volume of 18 mL, followed by addition of 150 μL of 10% APS and 60 μL of TEMED for polymerization.

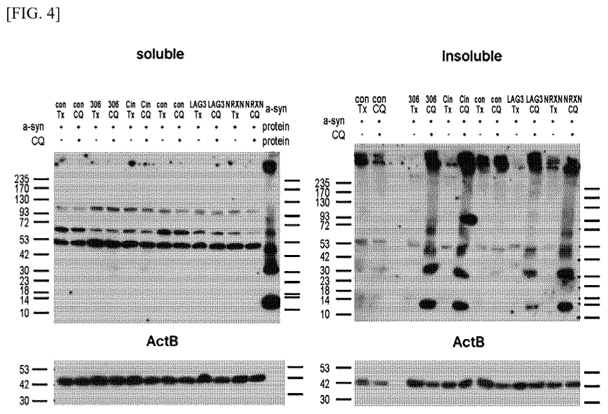

After electrophoresis, the proteins were transferred to a PVDF membrane and detected using an anti-αSyn antibody. In the soluble fraction, no difference in αSyn protein levels was observed regardless of chloroquine treatment. However, in the insoluble fraction, αSyn aggregates including a 15-kDa monomer, dimer, and oligomers, accumulated in chloroquine-treated cells (FIG. 4). These results indicates that cells expressing the CAR traffic αSyn aggregates to lysosomes for degradation.

### Example 6. Analysis of CAR Expression and αSyn Aggregate Clearance Ability in hPSC-Derived Astrocytes

Astrocytes were differentiated from hPSCs as described previously (J Cell Physiol. 236:7625 (2021) and Korean Patent No. 10-2534488). hPSC-derived neural progenitor cells (hPSC-NPCs) at passage 5 or lower were seeded into Matrigel-coated 6-well plates at 3×10⁴ cells/cm². The cells were then transduced with an rtTA-expressing lentivirus, an NFIB-expressing lentivirus, and the CAR-expressing lentivirus selected in Example 4 (MOI: 1.0 for each) in the presence of 1 μg/mL polybrene (Millipore Sigma). To increase transduction efficiency, the plate was centrifuged at 1,000×g for 1 hour at room temperature. After 18 hours of incubation, the virus-containing medium was replaced with fresh neural progenitor cell medium containing 2.5 μg/mL doxycycline (DOX) to induce NFIB expression. The day of DOX treatment was designated Day 0.

On Day 1, the medium was replaced with neural progenitor cell medium containing 10 ng/mL ciliary neurotrophic factor and 10 ng/mL BMP4 (Peprotech). On Day 3, the medium was replaced with a commercial astrocyte medium (ScienCell), and differentiation was continued until Day 14. To select virus-infected cells, 1.25 μg/mL puromycin was applied from Day 1 to Day 5.

On Day 14 of differentiation, cells were seeded in 24-well plates at 1×10⁵ cells per well, and 24 hours later, they were treated with Protonex Green-labeled αSyn aggregates at 1 μg/mL, prepared as described in Example 4. After 24 hours of treatment, the cells were harvested, washed twice with PBS, and the intracellular Protonex Green fluorescence was measured by flow cytometry. Analysis revealed a statistically significant increase in fluorescence intensity in hPSC-derived astrocytes expressing the CAR compared to the control group, indicating that the engineered cells can effectively traffic αSyn aggregates to lysosomes.

To efficiently express the CAR in astrocytes, the optimal timing for the CAR gene introduction was evaluated during the astrocyte differentiation process. During differentiation from hPSCs to astrocytes, a CAR-expressing lentivirus was introduced at three stages: the human embryonic stem cell stage (ES-CAR), the neural progenitor cell stage (NPC-CAR), and the astrocyte stage (AST-CAR).

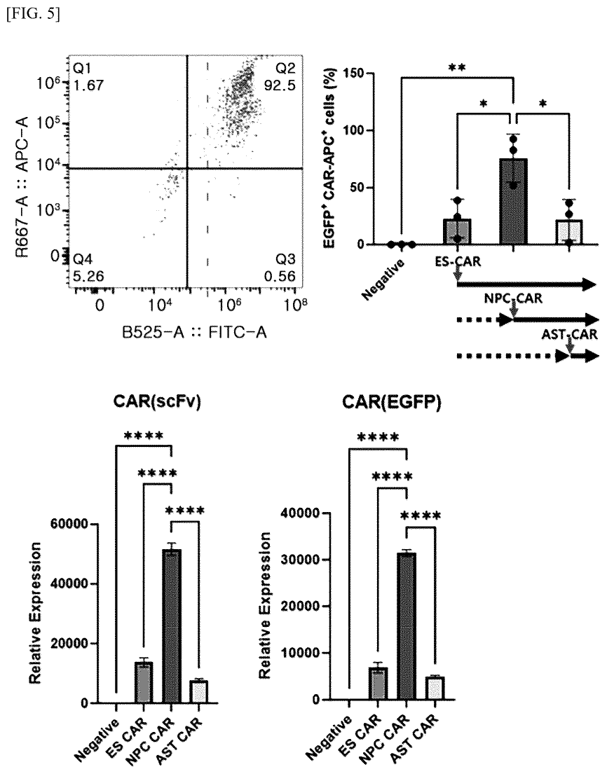

Flow cytometry analysis revealed that ES-CAR cells exhibited approximately 20% CAR expression, whereas NPC-CAR cells exhibited the highest expression rate at approximately 75%. AST-CAR cells exhibited approximately 25% expression. These results suggest that introduction of the CAR gene at the neural progenitor cell stage is the most efficient (FIG. 5).

Furthermore, qRT-PCR analysis of mRNA expression level demonstrated that NPC-CAR cells exhibited the highest mRNA levels. qRT-PCR analysis using scFv-specific primers revealed that NPC-CAR cells exhibited significantly higher mRNA levels than ES-CAR cells or AST-CAR cells. Specifically, NPC-CAR cells exhibited approximately three-fold higher mRNA levels than ES-CAR cells and approximately five-fold higher mRNA levels than AST-CAR cells. qRT-PCR analysis using EGFP-specific primers showed a similar trend, with the highest mRNA levels observed in NPC-CAR cells (FIG. 5). These results indicate that introduction of the CAR gene at the neural progenitor cell stage is optimal for efficient CAR expression, and further suggest that neural progenitor cells represent permissive cellular state for the introduction and expression of exogenous genes.

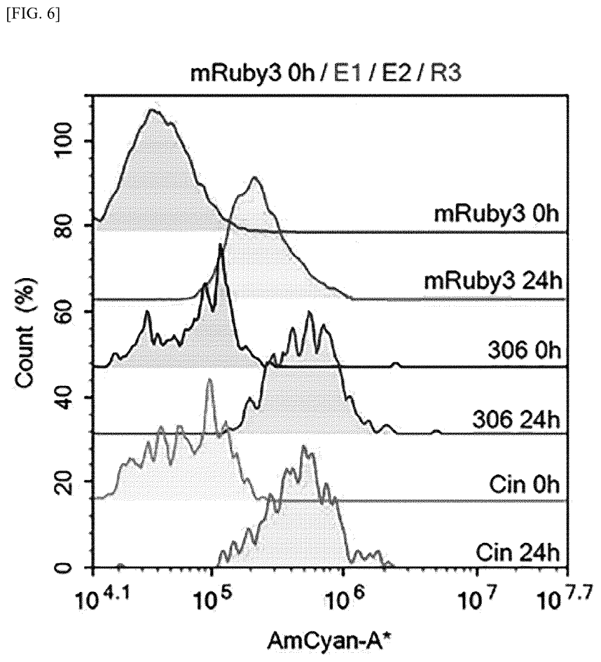

Finally, the functional activity of the CAR was evaluated in astrocytes differentiated from hPSCs. Astrocytes expressing a control construct (mRuby3) or CARs containing the 306C7B3 scFv or the Cinpanemab scFv were treated with Protonex Green-labeled αSyn aggregates, and time-dependent changes in intracellular fluorescence intensity were measured by flow cytometry (FIG. 6).

When fluorescence intensity was compared immediately after treatment (0 h) and at 24 hours post-treatment (24 h), a marked increase in fluorescence intensity was observed in astrocytes expressing the 306C7B3 sc Fv or Cinpanemab scFv CARs. These results indicate that, even in astrocytes differentiated from hPSCs, the CAR is capable of effectively capturing αSyn aggregates and trafficking them to lysosomes.

### Example 7. Evaluation of the Neuroprotective Effect of CAR-Expressing Astrocytes

To evaluate whether astrocytes expressing a CAR (CAR-Ast) can suppress neuronal damage, a co-culture experiment was performed using a dopaminergic neuronal injury model. Dopaminergic neurons differentiated from hPSCs were seeded in 24-well plates, and neuronal damage was induced by treatment with 6-hydroxydopamine (6-OHDA), a dopaminergic neuron-specific neurotoxin. Subsequently, fibroblasts (negative control) or CAR-Ast prepared in Example 6 were co-cultured to assess the effect on neuronal damage.

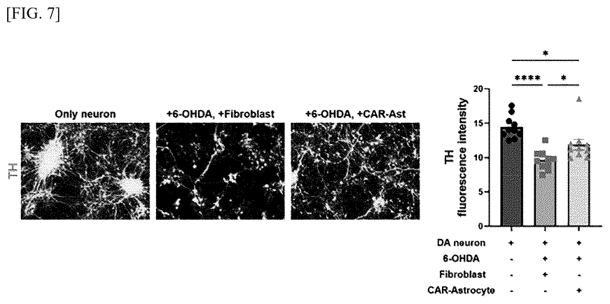

To evaluate the extent of neuronal damage, immunofluorescence staining for tyrosine hydroxylase (TH), a marker of dopaminergic neurons, was performed, and TH fluorescence intensity was quantitatively analyzed (FIG. 7).

Analysis showed that the highest TH fluorescence intensity was observed in the dopaminergic neuron-only culture group without 6-OHDA treatment (Only neuron). In the group co-cultured with fibroblasts after 6-OHDA treatment (+6-OHDA, +Fibroblast), TH fluorescence intensity was markedly reduced, confirming dopaminergic neuronal damage. In contrast, in the group co-cultured with CAR-expressing astrocytes after 6-OHDA treatment (+6-OHDA, +CAR-Ast), a significantly higher TH fluorescence intensity was observed compared to the fibroblast co-culture group (p<0.05).

These results indicate that astrocytes expressing the CAR can effectively suppress 6-OHDA-induced dopaminergic neuronal damage, suggesting their therapeutic applicability to dopaminergic neurodegenerative diseases such as Parkinson's disease.

### Example 8. Confirmation of the Effect of CAR-Expressing Astrocytes on Reducing αSyn Aggregates

To evaluate the therapeutic effect of CAR-expressing astrocytes on the intracellular accumulation of pathogenic αSyn, a key pathological hallmark of Parkinson's disease, an αSyn preformed fibril (PFF) model was used. hPSC-derived dopaminergic neurons were cultured alone or co-cultured with control astrocytes (mCherry-Ast) or CAR-Ast, and subsequently treated with PFFs to induce the accumulation of pathogenic αSyn.

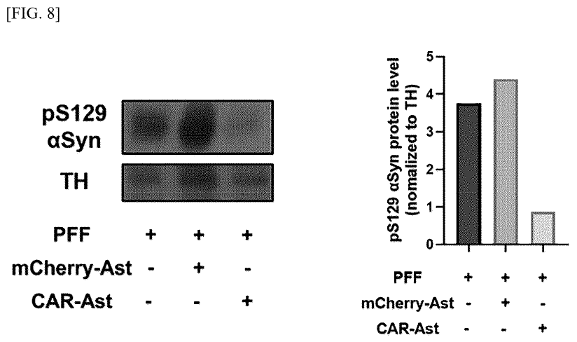

The level of αSyn phosphorylated at serine 129 residue (pS129 αSyn), a representative marker of pathogenic αSyn, was measured by Western blot analysis and quantified after normalization to tyrosine hydroxylase (TH) expression levels (FIG. 8). Analysis confirmed accumulation of pS129 αSyn in the group treated with PFF alone. In addition, the group co-cultured with mCherry-Ast showed a tendency toward further increased pS129 αSyn levels. In contrast, the group co-cultured with CAR-Ast exhibited a marked reduction in pS129 αSyn levels compared to the PFF-only treatment group, with levels reduced to approximately one-fifth of that observed in the control astrocyte co-culture group.

These results indicate that astrocytes expressing a CAR can effectively remove αSyn aggregates, thereby suppressing the formation of pathogenic pS129 αSyn within neurons. This further suggests that the CAR-mediated lysosomal trafficking of αSyn aggregates identified in Example 6 is also functional under pathological conditions, thereby conferring a therapeutic effect.

1. A recombinant glial cell comprising a chimeric antigen receptor, wherein the chimeric antigen receptor comprises any one of the amino acid sequences of SEQ ID NOs: 1 to 10.

2. The recombinant glial cell according to claim 1, wherein the glial cell is an astrocyte or a microglial cell.

3. The recombinant glial cell according to claim 1, wherein the recombinant glial cell comprises a chimeric antigen receptor composed of any one of the amino acid sequences of SEQ ID NOs: 2 to 4.

4. The recombinant glial cell according to claim 1, wherein the chimeric antigen receptor is introduced into the cell at a neural progenitor cell stage.

5. The recombinant glial cell according to claim 1, wherein the chimeric antigen receptor comprises a CD8 transmembrane domain.

6. The recombinant glial cell according to claim 1, wherein the chimeric antigen receptor comprises a MEGF10 intracellular domain.

7. The recombinant glial cell according to claim 1, wherein the chimeric antigen receptor is an alpha-synuclein aggregate-specific binding receptor.

8. A pharmaceutical composition for treating or preventing a neurodegenerative disease, comprising the recombinant glial cell of claim 1.

9. The pharmaceutical composition according to claim 8, wherein the neurodegenerative disease is Parkinson's disease.
